(19) United States
(12) Patent Application Publication
(10) Pub. No.: US 2010/0016436 A1
(43) Pub. Date: Jan. 21, 2010

(54) Transdermal Administration of Active Agents, in Particular Diclofenac

(75) Inventors: John Staniforth, Wiltshire (GB); Paul Goggom, Wiltshire (GB)

Correspondence Address: Davidson, Davidson & Kappel, LLC, 485 7th Avenue, 14th Floor, New York, NY, 10018, US

(73) Assignee: Pharmacodex Ltd., Chippenham, Wiltshire (GB)

(21) Appl. No.: 12/086,045

(22) PCT Filed: Dec. 7, 2006
(86) PCT No.: PCT/GB2006/050434

§ 371 Date: Sep. 16, 2009

(30) Foreign Application Priority Data
Dec. 7, 2005 (GB) 0524958.6

(51) Int. Cl. A61K 31/196; A61K 47/12; A61K 47/14; A61K 47/46

(52) U.S. Cl. 514/567; 514/784; 514/785; 514/786; 514/783

(57) ABSTRACT

The present invention relates to compositions for transdermal administration of therapeutic agents for providing a local and sustained therapeutic effect, wherein the extent of systemic administration can be controlled. In particular, the invention relates to spreadable compositions, or compositions which may be solid at a temperature of about 25° C. or less and have a softening point of not higher than 35° C., for use in the treatment of pain and/or inflammation or administration of a local anaesthetic, wherein transdermal administration of the therapeutic agent may be either rapid or sustained.

The present invention relates to compositions for transdermal administration of therapeutic agents.

Transdermal absorption is a well-recognized means of drug administration, which benefits from being a non-invasive and convenient way of medicating a patient. Transdermal absorption is a particularly useful means of drug administration for patients who find other methods difficult or unpleasant. For example, the young and the old can have difficulty with orally administered medications and may find injections particularly unpleasant. Children and patients with dementia can also be difficult to medicate due to lack of compliance. As such, transdermal administration of therapeutic agents could be a valuable method of administering a medication, especially in the young, the old or mentally impaired patients.

However, delivery of drugs across the skin has limitations. The outer most layer of the skin, the stratum corneum, is composed of dead keratin-rich cells (corneocytes) and a lipid matrix. The stratum corneum is 10-15 μm thick in adults and forms an effective barrier membrane that limits the type of molecules that can be absorbed by the skin, and also the rate of absorption. As such, transdermal administration of formulations containing therapeutic agents, has, to date, been limited.

Nevertheless, some therapeutic agents can be applied transdermally in order to provide local effects. For example, ibuprofen can be applied topically in the form of a gel in order to have an effect on local pain receptors within the skin. The gel is applied and rubbed into the affected area, for example, a painful joint. The gel is absorbed by the skin, and the ibuprofen acts upon local pain receptors to block the pain signal at its source. Further to this, some therapeutic agents can be administered transdermally in order to have a systemic effect, for example anti-nausea drugs such as scopolamine. In such cases, the formulation comprising the therapeutic agent is applied topically to the skin, whereupon the agent is absorbed through the skin and into the bloodstream.

Transdermal administration of therapeutic agents, whether for local or systemic use, relies upon the ability of the therapeutic agent to cross the stratum corneum, and the efficiency with which it does so. Permeation (or penetration) enhancers are frequently included in compositions for transdermal administration for this purpose, see for example U.S. Patent Application No. 2005-0074487. The therapeutic focus of compositions disclosed in the prior art tends to depend upon the location of receptors for the therapeutic agent in question. This location determines the extent to which the therapeutic agent needs to permeate the skin, and therefore the quantity of permeation enhancer required to ensure that the agent will reach the receptors.

As such, formulations in the prior art for transdermal administration of a therapeutic agent are generally unable to provide therapeutic local effects whilst also allowing the extent of the systemic effect of the therapeutic agent to be controlled. For example, topical application of ibuprofen gel for local administration may have a small systemic effect, as a result of a low concentration of the therapeutic agent, with respect to the amount of the agent applied to the skin, penetrating far enough through the skin to enter the bloodstream.

In some cases it may be desirable to minimize systemic administration of a therapeutic agent that has been transdermally administered to a patient in order to have a local effect. The systemic administration of some therapeutic agents can cause undesirable side effects. For example, ibuprofen is known to cause side effects such as dizziness and nausea in some patients when administered systemically, whilst local administration does not have this disadvantage.

In other situations, however, it is desirable to administer a therapeutic agent which has both local and systemic effects. For example, in a patient suffering from an inflamed joint, it may be beneficial for an anti-inflammatory agent to be topically applied to the joint in order to act at both local receptors and to enter the circulation and act systemically. In situations where systemic administration of a therapeutic agent is desired, it can be advantageous to administer the agent transdermally, rather than by other routes. The most common route of administering pharmaceutically active systemic agents is probably the oral route.

However, the oral administration of some active agents can cause side effects. For example, some agents, such as diclofenac, can cause gastric upset and gastric bleeding in some patients. Further to this, oral administration suffers from the disadvantage that there are variations between individuals in digestive breakdown time and efficiency can result in patients receiving variable doses of the agent and varying times to onset of the therapeutic effect. Systemic transdermal administration allows the therapeutic agent to enter the bloodstream through the skin, thereby initially bypassing the liver, stomach, and digestive system. Many side effects, such as irritation of the stomach lining, may therefore be diminished or eliminated. Further to this, by avoiding administration to the digestive tract, transdermal administration allows greater accuracy in achieving desired therapeutic levels of the agent in the bloodstream, thereby achieving optimal therapeutic effects, whilst minimizing potential side effects. First-pass metabolism of the therapeutic agent is also avoided.

Clearly, the benefits of transdermal administration as a means of drug delivery to a patient can only be fully realized if the local and systemic effects of the drug in question can be controlled.

An object of the present invention is, therefore, to provide a composition comprising one or more therapeutic agents and a pharmaceutically acceptable carrier for transdermal administration of the therapeutic agent, wherein the agent has a therapeutic local effect, and the extent of the systemic administration, and therefore the systemic therapeutic effect of the agent, can be controlled.

The effect of a topically applied composition comprising a therapeutic agent is governed by certain factors. These include, obviously, the location of receptors for the agent (i.e. whether receptors are present within the skin, or, for example located on viscera), and also factors such as the concentration of the agent within the formulation and the ease with which the formulation and/or agent is absorbed by the skin. This latter factor is influenced by the ease with which any carrier material present in the formulation is absorbed, or releases the therapeutic agent, and the presence and effectiveness of permeation enhancers in the formulation.

It has now been found that the systemic effect of transdermally administered therapeutic agents can be controlled by manipulating the factors mentioned above, through the careful selection of the types and quantities of carrier materials, therapeutic agents, permeation enhancers and solvents included in the composition.

The term “therapeutic agent”, “active agent” or “pharmaceutically active agent” denotes any active substance suitable for topical application and dermal administration to a patient (particularly a human patient) in a composition or product in accordance with the present invention. It is preferred that the therapeutic agent is an agent that is absorbed through the skin. Such agents include all of the drugs and classes of drugs referred to in the following passages, plus pharmaceutically acceptable equivalents thereof, such as their pharmaceutically acceptable salts, esters, prodrugs and active metabolites. Isomers of all disclosed agents are also encompassed by this disclosure.

The term “local administration” as used herein relates to the provision of a composition containing one or more therapeutic agents for application to the skin of a patient, wherein the therapeutic agent has an effect on the area of skin to which it is applied, upon receptors in the skin, and/or upon receptors in the layers of the skin within close proximity to the site of application of the composition, and wherein the therapeutic agent is not administered to the bloodstream.

The term “systemic administration” as used herein relates to the administration of the therapeutic agent to the blood stream.

As a result of the difficulties associated with transdermal administration of compositions containing therapeutic agents, known compositions for transdermal administration typically provide slow, sustained administration of the therapeutic agent through the skin over a period of time. In order to ensure that the compositions remain in contact with the skin for this gradual transdermal absorption, transdermal compositions are usually incorporated into a patch or plaster which is adhered to the skin. Such “medicinal plasters” are well known in the art. For example, patches containing anti-inflammatory agents have been used for patients suffering from joint inflammation (Gallacchi & Marcolongo, Drugs Exptl Clin Res 1993 XIX: 97-100).

Medicinal plasters have several advantages over other means of providing sustained levels of a drug in a patient. For example, medicinal plasters avoid the need for frequent dosing in order to achieve sustained drug effects. Medicinal plasters also provide an easy and non-invasive way of administering a drug.

There are currently three major types of transdermal patch systems, namely membrane-controlled systems, adhesive diffusion-controlled systems or matrix systems.

The membrane-controlled system typically consists of four layers: an impermeable backing layer, a polymer layer that serves as a drug reservoir, a rate-controlling microporous membrane, and an adhesive. The drug reservoir comprises the therapeutic agent and liquid excipients that encourage absorption of the drug across the skin. Upon application to the skin, the drug diffuses through the membrane and then passes through the adhesive before reaching the skin. The drug release rate is constant, so, in order to provide the most efficient method of administration, the release rate must be maintained at a level just below the saturation limit of the skin. U.S. Pat. No. 5,683,712 discloses an example of a membrane-controlled system for transdermal administration of homeopathic drugs, wherein a micro-porous membrane is provided for controlling the release of the drug, with a gel containing the drug scattered within the membrane.

The adhesive diffusion-controlled system is very similar to the membrane-controlled system except that the rate-controlling microporous membrane is absent. The system consists instead of an impermeable plastic barrier, a drug reservoir and one or more rate controlling adhesive layers next to the skin. DE 19849823 discloses an example if an adhesive diffusion-controlled system, the medicinal plaster comprising a backing layer, a reservoir containing the active agent and an adhesive layer overlying the reservoir layer which has non-adhesive regions allowing passage of the active agent on contact with the skin.

In the matrix system, the drug reservoir is in direct contact with the skin. As such, the rate of diffusion of the drug is dependent upon the absorption rate of the skin. The system may comprise an impermeable backing attached to a drug reservoir consisting of a hydrophilic or hydrophobic polymer containing the dispersed drug. WO 87/00042 describes such a system, for transdermal administration of verapamil at a sustained, substantially uniform rate over an extended period of time. Alternatively, the system may comprise a backing, and an adhesive layer which serves a dual purpose as both the adhesive and the drug reservoir. An example of such a system is described in WO 00/02539, which relates to a plaster containing a non-steroidal anti-rheumatic agent.

However, the known plaster systems suffer from several disadvantages. Each system entails release of the drug from a material reservoir, which is, typically, a tissue tolerant polymer. As such, the surface area for absorption of the drug is, in each case, limited to the surface area of the material that is in contact with the skin. The production of membrane, matrix and adhesion-controlled systems is also technically costly and requires special apparatus.

Further disadvantages are associated with the use of adhesive plasters for transdermal administration of therapeutic agents. For example, the adhesive used on plasters has to be compatible with the therapeutic agent and other constituents of the composition used with the plaster. This means that such plasters can be expensive and that not all therapeutic agents are suitable for inclusion in them. Additionally, the application of a plaster can be both impractical and undesirable, especially if the plaster is attached to the skin for a prolonged period of time.

An object of the present invention is, therefore, to provide a composition comprising one or more therapeutic agents and a pharmaceutically acceptable carrier, which provides a relatively simple and inexpensive, yet effective means of transdermal administration of the therapeutic agent to provide a local therapeutic effect, and wherein the extent of the systemic administration, and therefore the systemic therapeutic effect of the agent, can be controlled.

A further object of the present invention is to provide compositions which may be incorporated into a medicinal plaster or patch, as well as to provide compositions which do not need to be used in conjunction with a covering which is to be affixed to the skin using an adhesive.

Further to this, some conditions would benefit from rapid administration of a therapeutic agent. For example, in situations where rapid alleviation of symptoms is desirable, such as in patients suffering from pain or inflammation. Common routes of administering pharmaceutically active agents for rapid effect include, for example, injection. However, these routes of administration are frequently not intended to provide a local effect. Transdermal absorption has not been considered, until now, to be a feasible method of providing rapid systemic drug administration, due to the limitations on absorption rate imposed by the stratum corneum.

Thus, a further object of the present invention is to provide a composition comprising one or more therapeutic agents and a pharmaceutically acceptable carrier, which provides a means of rapid transdermal administration of the therapeutic agent, wherein the agent has a therapeutic local effect, and the extent of the systemic administration, and therefore the systemic therapeutic effect of the agent, can be controlled.

In particular, an object of the present invention is to provide a composition which can simply be spread over an area of skin, from where it is rapidly absorbed, resulting in quick delivery of the therapeutic agent, thereby providing a rapid therapeutic effect. Such a composition would be of enormous benefit in situations where rapid alleviation of symptoms is desirable, and where it is undesirable for the patient to have to use an invasive means of drug administration.

The compositions provided by the present invention achieve the object of rapid or sustained transdermal absorption by the use of appropriate compositions which control the release of the active agent when applied to the skin, and therefore the delivery kinetics. This means that the rate of delivery of an active agent to the skin from a composition according to the present invention can be altered in accordance with therapeutic requirements. The rate of delivery of the active agent to the skin can be modified by altering the affinity of the active agent for the carrier material compared to the affinity of the active agent for transportation through the stratum corneum. This is achieved by the preparation and use of particular carrier materials in the composition, which are tailored to the active agents in question, in order to alter the hydrophilicity of the composition. Using this approach, the solvent properties of the composition, and therefore the solubility profile of the active agent can be modified, which means that the rate of diffusion of the active agent can be controlled.

According to a first aspect of the present invention, there is provided a pharmaceutical composition comprising a therapeutic agent and a pharmaceutically acceptable carrier, the composition being suitable for topical application to the skin of a mammalian patient, resulting in transdermal administration of the therapeutic agent, wherein transdermal administration provides a local therapeutic effect.

In certain embodiments, the pharmaceutical composition according to the first aspect of the present invention also provides therapeutic plasma concentrations of the therapeutic agent upon topical application, thereby further producing a therapeutic systemic effect. Preferably, the plasma concentration levels of the therapeutic agent are between the minimum and maximum therapeutically effective levels.

The pharmaceutical compositions which provide both local and systemic effects according to the present invention are for use in therapy or prophylaxis. In preferred embodiments, compositions according to the first aspect of the present invention are not used for treating acne, or for providing hormone-replacement therapy, nicotine-replacement therapy or contraception.

Therapeutic agents which may advantageously be included in the compositions of the present invention include those which are usually administered for the treatment of pain and/or inflammation, for example, ibuprofen, aceclofenac, acemetacin, azapropazone, celecoxib, dexketoprofen, diclofenac, diflunisal, etodolac, etoricoxib, fenbufen, fenoprofen, flurbiprofen, indometacin, ketoprofen, mefenamic acid, meloxicam, nabumetone, naproxen, piroxicam, rofecoxib, sulindac, tenoxicam, tiaprofenic acid, valdecoxib, glyceryl trinitrate, opioids such as fentanyl buprenorphine, salicylic acid and related salicylates; or any agent used as a local anaesthetic, for example lignocaine, lidocaine and prilocaine, bupivacaine, levobupivacaine, procaine, ropivacaine, tetracaine, benzocaine or amethocaine.

Preferably, the therapeutic agents included in the compositions of the present invention primarily have a local therapeutic effect.

Pharmaceutical compositions according to the present invention may comprise more than one therapeutic agent, provided that they are compatible with one another under conditions of storage and use.

In some embodiments, the compositions according to the first aspect of the invention comprise a means of substantially occluding the therapeutic agent from the air following application to the skin. The occlusive means is provided by the use of appropriate quantities of wax, oil or fat in the carrier material of the composition.

Such compositions comprising a means of occlusion are able to provide the advantages associated with the use of medicinal patches and plasters, without the disadvantages. In particular, the compositions comprising an occluding means increase hydration of the skin beneath the occlusion and cause dilatation of pores in the skin, thereby enhancing the absorption of the therapeutic agent from the composition. The occluding means also serves to protect the composition from the environment, ensuring that the composition does not get rubbed off, which may result in incomplete administration of the dose of therapeutic agent provided by the composition.

In certain embodiments, the compositions according to the first aspect of the present invention are spreadable, for example, they are provided in the form of a cream, ointment or gel. Such spreadable compositions have the advantage that they are easily applied and rubbed into the skin in order to aid absorption.

In embodiments of the present invention wherein the pharmaceutical composition is provided as a spreadable composition, the carrier medium should allow the therapeutic agent to be carried in a stable manner. The carrier medium may have favourable organoleptic properties, for example, the composition may be water-based so as to have a non-oily feel upon application to the skin. A carrier medium should be compatible with the therapeutic agent, affording the therapeutic agent chemical stability. The make-up of the compositions of the present invention should be designed to encourage the flux of drug from the composition and through the stratum corneum.

Components which are commonly used as a base for creams, ointments or gels may be used as a carrier medium in the compositions according to the present invention. These components include: water; hydrocarbon oils and waxes; silicone oils; vegetable, animal or marine fats or oils; glycerides (such as, for example, or more glycerol esters of saturated fatty acids or polyglycolysed glycerides, cocoa butter, theobroma or the like) or glyceride derivatives; high molecular weight polyethylene glycol, polyoxyethylene, lanolin and derivatives thereof; fatty acids, fatty alcohols or fatty esters (including, for example, caprylic acid, caprylic triglyceride or the like); lecithin; polyhydric alcohols or esters; wax esters; sterols; phospholipids and the like. Thickening agents such as gums or other forms of hydrophilic colloids may be included. The carrier medium may comprise more than one base component.

Where the pharmaceutical composition is a cream, the carrier medium may comprise substantially more oil based components than water. Where the pharmaceutical composition is an ointment, the carrier medium may comprise substantially more water than oil based components. Where the pharmaceutical composition is a gel, the carrier medium may substantially comprise water.

Where the composition is provided as a cream, ointment or gel, it is possible to accurately control the dose to be applied to the skin of the patient by providing one or more unit or measured doses of the spreadable composition. This helps to ensure that the patient receives an accurate, predetermined dose of therapeutic agent. In the present invention, such unit or measured doses may be packaged individually, for example in containers such as tubes or sachets. Alternatively, the compositions of the present invention may be dispensed by a device which is capable of dispensing an accurate, predetermined amount.

In alternative embodiments of the present invention, compositions for rapid transdermal administration have a substantially solid form at a temperature of about 25° C. or less, and a softening point of not higher than the skin temperature of a mammalian patient as substantially hereinafter described. More particularly, the pharmaceutical composition is in a substantially solid form during storage (at a temperature of or below 25° C.). However, following application of the composition to an area of the skin of a mammalian patient, the solid composition softens to a consistency that can be substantially absorbed by the area of skin so as to effect transdermal administration of the therapeutic agent to the mammalian patient.

The provision of compositions for rapid transdermal administration which are solid at temperatures of about 25° C. or less allows ease of handling and application of the composition. Softening of the composition of the present invention upon contact with the skin, however, allows the composition to enjoy the favourable absorption properties and ease of spreading associated with non-solid compositions. Spreading of the softened composition upon contact with the skin also facilitates the passage of the active agent from the composition through the stratum corneum, by increasing the area of the composition in contact with the skin, thereby providing a greater surface area for the active agent to diffuse through the stratum corneum in accordance with the classical diffusion theory.

The carrier medium used in the compositions of the present invention which soften upon contact with skin is selected to be substantially solid at a temperature of about 25° C. or less, and to soften to a consistency that allows for substantially complete absorption of the one or more therapeutic agents by the area of skin of the mammalian patient at a temperature of above 25° C. It is preferred that the carrier medium softens, and advantageously may be converted to a spreading consistency, at a temperature in the range of 30 to 35° C. or up to around 37° C.

The compositions of the present invention are preferably provided for application to a human patient. In such embodiments, the compositions preferably have a softening point which is not higher than the normal temperature at the skin surface (skin temperature) of a human. This temperature is typically not higher than about 35° C. In certain embodiments, the composition has a softening point from about 25° C. to about 35° C., or from about 30° C. to not higher than about 35° C.

In one embodiment of the invention, the pharmaceutical composition is solid at a temperature of about 25° C. or less and has a softening point of not higher than 35° C., such that when the composition is placed in continuous contact with the skin of a mammalian patient, it is softened to a consistency to effect substantial application of the therapeutic agent onto a desired skin area of the mammalian patient within a time period of less than 10 minutes.

This allows for substantially complete absorption of the composition over the area of skin, so as to effect substantially complete administration of the therapeutic agent to the mammalian patient.

The term “softening point” as used herein refers to a temperature at which a substantially solid dosage form starts to soften to a consistency that can be absorbed by the skin of a patient, so as to allow transdermal absorption of the therapeutic agent present in the composition.

The softening point of a substantially solid dosage form of a pharmaceutical composition according to the first aspect of the present invention can be determined visibly as the temperature at which the substantially solid dosage form starts to soften to a consistency that can be spread and absorbed by the skin of a patient and as such can advantageously be substantially completely absorbed by the skin of the patient so as to leave little or no undesirable residue on the skin of a patient.

Alternatively, the softening point of a substantially solid dosage form of a pharmaceutical composition according to the first aspect of the present invention can be determined using a TA-XT2 texture analyser (Stable MicroSystems Ltd., UK), suitably equipped with a 5 kg load cell. The equipment is enclosed in a temperature controlled chamber (capable of operating in the region of 60° C. to 200° C.). A tablet or other substantially solid dosage form according to the present invention may be enclosed in the chamber at the specified temperature for a time of at least 10 minutes. A 3 mm flat faced probe is pushed into the tablet or other substantially solid dosage form according to the present invention for a distance of 1 mm at a speed of 0.1 mm/sec. Measurements can be repeated at temperature increments of 1° C. and, at the temperature at which the peak force of resistance recorded (as measured by Texture Exceed software) falls to below 50% of that for a “solid” tablet or other substantially solid dosage form according to the present invention, the tablet or other dosage form is deemed to have “softened”.

The term “spreading point” as used herein refers to a temperature at which the composition has a spreading consistency. For example, the composition may flow under its own weight or at least can be spread upon the skin of a mammalian patient, for example, using finger pressure.

The mobility of a spreading composition may promote the absorption of the therapeutic agent into the skin by allowing movement of the therapeutic agent towards the skin, for example, by diffusion. The spreading point of a preparation may be measured using the TA-XT2 texture analyser mentioned above in relation to measurement of softening point and with this analyser the spreading point of a composition is the temperature at which outward flow of the composition is first observed on advance of the flat faced probe into the preparation.

In another embodiment, the pharmaceutical composition suitable for topical administration, preferably to a mammal, comprises one or more therapeutic agents and a carrier medium, wherein said preparation has a softening point of not higher than skin temperature of a mammalian patient, said composition having an aspect ratio (wall:face) of less than 1:1.

In another embodiment, the pharmaceutical composition for topical administration, preferably to a mammal, comprises a compacted granulate including one or more therapeutic agents and a pharmaceutically acceptable carrier, said compacted granulate having a softening point of not higher than skin temperature of the intended subject, preferably a mammalian patient.

In certain embodiments, the composition, which is solid prior to administration by application to the skin, has a shape to facilitate the topical application. For example, the composition can have: at least one flat surface; at least one concave surface; at least one convex surface; two flat surfaces; two concave surfaces; or two convex surfaces. The composition may be in the form of a standard tablet, spherical or half-spherical. Bullet shaped and conical shaped compositions are not preferred in the present invention.

In preferred embodiments, the compositions of the present invention have a total weight of from about 50 mg to less than 1 g, preferably from about 100 mg to about 900 mg and more preferably from about 250 mg to about 750 mg. The compositions of the present invention can have a total weight of 1 g or greater, if desired.

In certain embodiments of the present invention, the compositions are provided as a solid unit dosage form and comprise a therapeutically effective amount of at least one therapeutic agent for topical application to a mammalian patient. The unit dosage form is a solid during final manufacture and has, upon application to an area of skin of said mammalian patient, a spreading consistency suitable for application to said area of skin. Unit dosage forms may be packaged individually, for example, in a plastic container having a removable or breakable enclosure for dispensing said unit dosage form.

Typically, the substantially solid unit dosage form is provided in the form of a tablet or of a rolled preparation, for example, a pill or the like.

In certain embodiments, the dosage form can be a plurality of substantially discrete substantially solid particles comprising one or more therapeutic agents admixed with a pharmaceutically acceptable carrier, said particles having a softening point of about 30° C. to about 35° C. The particles can be enclosed in a sachet, a capsule or a device suitable to dispense an individual dose of the particles.

More particularly, it is preferred that the shape and configuration of the substantially solid dosage form is determined by the softening point of the composition and/or the carrier medium. It may be preferred that a substantially solid dosage form according to the present invention comprises a substantially unitary form; alternatively, it may comprise a plurality of discrete particles (such as a plurality of granules or the like) that can be absorbed by the skin of a mammalian patient. Preferably, the plurality of substantially discrete particles are provided in a sealed member (such as a capsule, sachet, blister package or the like) from which they are dispensed and applied to the skin of a patient.

Any component commonly used for suppositories can be used as carriers in the compositions of the present invention which soften upon application to the skin. These components include those derived from mammalian, vegetable or mineral origins, and materials partially or totally synthesized. Specific examples of such carriers include oils and fats of mammalians or vegetable origin, such as olive oil, corn oil, castor oil, cottonseed oil, wheat germ oil, cacao butter, hydrogenated oils, etc.; hydrocarbons, such as squalane, petrolatum, solid paraffin, liquid paraffin, etc.; and waxes, such as jojoba oil, carnauba wax, bees wax, lanolin, etc. Examples of partially or totally synthesized fatty acid esters include glycerol, mono-, di-, or triglycerides of medium or higher fatty acid, such as saturated linear fatty acid, for example lauric acid, myristic acid, palmitic acid, stearic acid, etc., or unsaturated linear fatty acids, for example oleic acid, linoleic acid, linolenic acid, etc. Commercially available carriers which are suitable include Witepsol (manufactured by Dynamit Nobel), Pharmasol (manufactured by Nippon Oil and Fats Co.), Isocacao (manufactured by Kao Corp.), SB (manufactured by Taiyo Oil and Fats Co.), Novata (manufactured by Henkel), Suppocire (manufactured by Gattefosse Co.), and the like. Examples of other synthetic products include polyethylene glycol, for example, macrogole, setomacrogole, etc., as well as derivatives thereof, for example, setomacrogol.

In order to obtain the desired softening point of the compositions of the present invention, different carriers can, if necessary, be combined in order to increase or decrease the softening point to obtain a suitable product. For example, in order to decrease the softening point, a plasticizer can be added, e.g., glyceryl monostearate, myristyl alcohol, polysorbate 80, propylene glycol or combinations thereof. In order to increase the softening point, a hardener can be added, e. g., beeswax, cetyl alcohol, stearic acid, stearyl alcohol, aluminium monostearate, aluminium distearate, aluminium tristearate, bentonite, magnesium stearate, colloidal silicon dioxide or combinations thereof.

A carrier for use according to the present invention may comprise any ingredient suitable for use in a pharmaceutical composition and possessing the desired properties for enabling topical administration of a dose of at least one therapeutic agent, provided that it is suitable for topical application and transdermal administration. For example, the carrier may include a cellulose or one or more ingredients selected from the group consisting of ingredients of the type suitable for use in suppositories including, for example, one or more glycerides (such as, for example, one or more glycerol esters of saturated fatty acids or one or more polyglycolysed glycerides, cocoa butter, theobroma or the like), one or more high molecular weight polyethylene glycol, one or more polyoxyethylene, lanolin and derivatives thereof, and one or more fatty acids, fatty alcohols, fatty acid esters (including, for example, caprylic acid, caprylic triglyceride or the like), and any of the preceding ingredients can be optionally mixed with one or more organic oils (including, for example hydrogenated vegetable oils) or the like.

It is often preferred that a carrier employed in a pharmaceutical composition according to the present invention comprises, and more preferably consists essentially of, one or more glycerides, including, in particular, one or more glycerol esters of C8-C18 fatty acids or one or more polyglycolysed glycerides.

Suitably, the carrier of a pharmaceutical composition according to the present invention comprises, or consists essentially of, a mixture of glycerides, where the glycerides can be one or more mono-, di- or tri-glycerides, optionally wherein the glycerides comprise glycerol esters of C12-C18 fatty acids. In one embodiment, the glyceride mixture is a Witepsol grade product. More particularly, the carrier may comprise, or consist essentially of, a Witepsol grade product available under any of the trade marks Witepsol H5, Witepsol H15, Witepsol H32, Witepsol S51, Witepsol S55, Witepsol S58, Witepsol W25 and Witepsol W32. In a particularly preferred embodiment, the pharmaceutical compositions according to the present invention include carriers which are Witepsol grade products available under any of the following trade marks Witepsol H5, Witepsol H15, Witepsol S51 and Witepsol S55. The Witepsol grade product available under the trade mark Witepsol H15 is particularly suitable.

In a particular embodiment of the present invention, the carrier employed in the compositions consists essentially of a Witepsol grade product substantially as described above.

Alternatively, the carrier comprises, or consists essentially of, a mixture of glycerides, where the glycerides can be selected from the group consisting of mono-, di- and tri-glycerides, the glycerides comprising glycerol esters of Cg-Cie fatty acids or one or more polyglycolysed glycerides. In one embodiment, glyceride mixtures available under the trade marks Gelucire or Suppocire are used, such as any of the following: Gelucire 33/01, Gelucire 39/01, Gelucire 43/01, Gelucire 44/14, or any of the Suppocire Standard type, Suppocire N type or Suppocire P type products.

Alternatively, the carrier used in a pharmaceutical composition according to the present invention comprises, or consists essentially of, cocoa butter.

Methods of preparing the softening compositions referred to above are disclosed in WO 02/00203 A1.

The technology disclosed herein can be applied to pharmaceutical compositions for providing rapid transdermal administration of the therapeutic agent. The term “rapid” as used herein relates to the absorption of a therapeutic agent into the bloodstream within, for example, in less than about 20, 15, 10, 5, 4, 3 or 2 minutes, or less than about 1 minute or 0.5 minutes from application to the skin.

In a preferred embodiment of the present invention, the composition provides transdermal administration of the therapeutic agent within a period of 20 minutes from application to the skin.

Preferably, topical application of the composition to the skin of a mammalian patient results in transdermal administration of the therapeutic agent within a period of less than 15, 10, 5, 4, 3 or 2 minutes, or less than 1 minute or 0.5 minutes from application to the skin.

The technology disclosed herein can also be applied to pharmaceutical compositions for providing sustained transdermal administration of the therapeutic agent. The term “sustained” as used herein relates to the provision of a composition from which a therapeutic agent can be absorbed by the skin over a period of time, for example, greater than about 30 minutes, or greater than about 1, 2, 4, 8, 12, 16, 20, 24, 26, 28, 32, 36, 48 or 72 hours.

Thus, in a yet further embodiment of the present invention, the topical application of the composition provides transdermal administration of the therapeutic agent over a period greater than 30 minutes from application to the skin.

Preferably, transdermal administration of the therapeutic agent is provided over a period greater than about 30 minutes, or greater than about 1, 2, 4, 8, 12, 16, 20, 24, 26, 28, 32, 36, 48 or 72 hours from application to the skin.

The sustained therapeutic effect may be local and/or systemic in nature.

In further embodiments of the invention, the compositions are incorporated into medicinal plasters or patches which include a covering layer which covers the composition and is to be affixed to the skin of a patient, preferably by an adhesive which is located around the edge of the covering layer. Such plasters or patches will cause dilatation of the pores, and increase hydration of, the skin beneath the covering layer when they are applied to the skin, thereby enhancing absorption of the therapeutic agent from the composition. The covering layer also serves to protect the composition from the environment, ensuring that the composition does not get rubbed off, which may result in incomplete administration of the dose of therapeutic agent provided by the composition.

Plasters according to the present invention avoid several of the disadvantages associated with medicinal plasters known in the art. For example, in plasters according to the present invention, the rate of delivery of the drug to the desired receptor site is controlled as a result of the composition itself, and does not, therefore, rely upon the use of rate-controlling adhesive layers, membranes, matrices, or adhesion controlled system, which can be technically costly and require special apparatus to make. Further to this, in such embodiments of the present invention, the purpose of the plaster is merely to hold the composition in contact with the skin, and as such, the plaster is prepared for use by simply locating a desired quantity of a composition, prepared in accordance with the first aspect of the present invention, under a covering with an adhesive edge. Unlike plasters known in the art, no special methods of manufacture, which can again be costly, are required in order to incorporate the composition into a rate-controlling layer. The compositions incorporated in the plasters according to the present invention are preferably in the form of a tablet, and are preferably made on a tabletting machine.

This is, again, a very different method of producing plasters from the way in which known medicinal plasters are made.

The incorporation of the compositions of the present invention into such plasters or patches is particularly desirable where the compositions are used to administer therapeutic agents over a prolonged period of time. For example, slow or sustained release of agents such as water-soluble vitamins and hormones may be desired over a period of months.

The adhesive used in the medicinal plasters or patches according to the present invention should be compatible with the therapeutic agent.

In certain embodiments, the medicinal plaster or patch further comprises an absorbent material, which is located on the same side of the covering layer as the pharmaceutical composition. In such embodiments, the pharmaceutical composition melts upon contact with the skin and is absorbed by the absorbent material. This avoids the formation of a molten reservoir or pool of non-solid composition under the covering layer, which might otherwise seep out from the plaster or patch over time. The absorbent material may be any substance suitable for the absorption of the pharmaceutical composition, including, for example, felt, cotton wool or polyurethane foam.

In an alternative embodiment, the composition of the present invention comprises one or more film-forming materials, which are capable of forming a protective layer when the composition is applied to the skin of a patient. These film-forming materials do not rapidly melt upon application to the skin, but rather form a protective layer, thereby allowing the therapeutic agent to be absorbed across the skin from beneath the protective layer.

The inclusion of a film-forming layer in the composition avoids the need for a plaster, or other form of adhesive covering member. This can be advantageous, as discussed above, in avoiding the difficulties associated with the use of plasters and adhesives. Inclusion of a film-forming layer would be of particular use when the composition comprises a carrier material and/or an active agent which may volatise or evaporate at skin temperatures.

Compositions of the present invention should be stored at temperatures of about 25° C. or less, in accordance with storage conditions for most pharmaceutical compositions and formulations.

Preferably, the carrier medium in compositions according to the present invention constitutes not less than about 60%, more preferably not less than about 80% and even more preferably not less than about 90%, by weight based on the weight of the pharmaceutical composition.

Preferably, the therapeutic agent or agents in compositions according to the present invention are present in the pharmaceutical composition in a therapeutically effective concentration of at least 0.01% by weight based on the weight of the pharmaceutical composition.

Pharmaceutical compositions according to the present invention may further comprise, where appropriate, additional ingredients such as one or more penetration enhancers (which may be surfactants, alcohols, esters, glycols or the like or any other suitable penetration enhancer), humectants, surfactants (which may be cationic, non-ionic, anionic or polymeric), emulsifiers, antioxidants, preservatives, clays, antifoaming agents, spreading agents, emollients, barriers, solubilising agents for the therapeutic agent and the like.

Pharmaceutical compositions according to the present invention may also comprise solvents, such as ethanol, menthol, thymol, eucalyptol, eucalyptus oil, benzyl alcohol, isopropyl alcohol, propylene glycol, methylated spirit, phenol, cyclodextrins, ethyl oleate, eugenol, glycerol, levomenol, monoethanolamine oleate, myristyl alcohol, octyldodecanol, methyl alcohol, coconut oil or silicone oil.

The presence of solvents in compositions according to the present invention allows controlled transdermal administration of the therapeutic agent. The extent to which, and speed with which local and systemic administration of a therapeutic agent from a topically applied composition occurs is associated with the depth and rate of penetration of the therapeutic agent through the skin. The compositions provided by the present invention achieve the object of local, and controlled systemic transdermal administration by the use of particular carrier materials in the composition in order to alter the hydrophilicity, as hereinbefore discussed, and the inclusion of solvents. The presence of solvents in compositions according to the present invention aids solubilization of the drug within the composition. Solvents for use in the present invention are also chosen in accordance with their ability to cross or bridge the stratum corneum, and tight junctions between the corneocytes within the stratum corneum in particular. The presence of solvents in the present invention thus alters the rate of transdermal absorption, and the depth of penetration of the therapeutic agent by solubilizing the agent, and effecting diffusion of the agent through the stratum corneum. The rate and depth of delivery of the therapeutic agent to the bloodstream and local receptors, and therefore the effect of the agent can thus be modified in compositions according to the present invention by the selection and use of particular types and quantities of solvents.

Pharmaceutical compositions according to the present invention may further comprise organoleptic agents to improve the organoleptic properties of the composition. Such agents include almond oil, glycerol linseed oil, monoethanolamine oleate, grape oil, mace oil, isopropyl myristate, isopropyl palmitate, palm kernel oil, theobroma oil, wool alcohols. The inclusion of organoleptic agents can be used, for example, to enhance the feel of the composition, which can improve patient compliance. In addition, such agents can have a perceived cooling effect, which can provide a positive psychological effect, particularly, for example, in the case of application to an inflamed joint.

Pharmaceutical compositions according to the present invention may further comprise sensory cues, such as anise oil, citronella oil, clove oil, eucalyptol, eucalyptus oil, eugenol, juniper oil, lemon grass oil, lemon oil, terpeneless lemon oil, melaleuca oil, neroli oil, nutmeg oil, olive oil, orange oil, terpeneless orange oil, poppy seed oil, pine oil, rose oil, sage oil, spearmint oil, lavender oil, thyme oil, vanillin.

The inclusion of such cues in the composition can provide the patient with pleasant sensory feedback upon use, allows the patient and/or person applying the formulation to recognize that administration has occurred, and may aid recollection of administration. Such factors can improve patient compliance and provide a positive psychological effect.

Pharmaceutical compositions according to the present invention may further comprise insect repellents such as citronella or lemon grass.

In some embodiments of the present invention, the compositions are substantially free of penetration enhancers. In such embodiments, the compositions are preferably prepared using a process carried out under aseptic conditions.

The use of preservatives can be undesirable, as they may provoke allergic reactions in susceptible patients, and the present invention may be advantageous in avoiding or reducing the risk of such allergic reactions. Preservatives that have been associated with allergic reactions include chlorocresol, hydroxybenzoates (parabens), polysorbates, sorbic acid and the like, and these preservatives are included in a large number of known topical compositions, including, for example, compositions available under any of the following trade marks: Drapolene, Medicaid, Siopel, Sprilon, Eurax, Efcortelan, Mildison, Fucidin H, Nystaform, Quinocort, Terra-Cortril Nystatin, Timodine, Locoid, Locoid Crelo, Modrasone, Propaderm, Betnovate, Betnovate RD, Diprosone, Dermovate, Eumovate, Trimovate, Nerisone, Haelan, Synalar, Ultralanum Plain, Zorac, Carbo-Dome, Exorex, Differin and Exelderm.

In a particularly preferred embodiment of the present invention, the pharmaceutical compositions are substantially free of the types of preservative generally included in compositions intended for dermal or transdermal administration, or at least they include such preservatives in amounts that are less than those generally required in compositions intended for dermal or transdermal administration, or they include such preservatives in amounts that generally do not provoke substantial allergic reactions in susceptible patients, substantially as hereinafter described.

The preservatives generally employed in compositions intended for dermal or transdermal administration are included to prevent or reduce contamination of such compositions. Contamination is a particular problem where a composition is repeatedly exposed to the atmosphere or is repeatedly handled. Preservatives may not be required in compositions of the present invention where the compositions are in the form of unit doses, especially if these doses are individually packaged.

Pharmaceutical compositions according to the present invention may, however, comprise one or more preservatives, such as phenoxyethanol or the like, that are included typically to substantially prevent contamination of the compositions according to the present invention during manufacture but are not generally of the type employed to prevent infection due to manual application as hereinbefore described.

In further embodiments of the present invention, the compositions are substantially free of antioxidants. In such cases, it is preferred that the compositions are packaged in a substantially inert atmosphere, such as nitrogen or the like.

The use of antioxidants can provoke allergic reactions in susceptible patients and the present invention may be advantageous in avoiding or reducing the risk of such allergic reactions in susceptible patients. Antioxidants that have been associated with allergic reactions include butylated hydroxyanisole, butylated hydroxytoluene and the like, and are known to be available in prior art topical compositions, such as those compositions available under any of the trade marks Imuderm, Siopel and the like.

According to a second aspect of the present invention, an applicator is provided for applying compositions according to the first aspect of the present invention to the skin of a patient.

In one embodiment, the applicator is a pad, sponge, bar, brush, cotton ball or glove.

In another embodiment, the applicator comprises a receiving means for receiving and carrying a unit or measured dose of the pharmaceutical composition and a grip for enabling a user to hold and manipulate the applicator. The grip and receiving means may be arranged such that a user holding the applicator by the grip is protected from inadvertent contact with the composition, a unit or measured dose of which is carried by the receiving means.

In a preferred embodiment, the composition is substantially solid at temperatures below 25° C. and softens upon contact with the skin of the patient. The composition may be in the form of a solid unit dosage form.

In certain embodiments, the applicator may comprise a first portion coupled to the unit dosage form and a second portion configured for being held by a user.

In alternative embodiments, the applicator may also include an intermediate member attached to the composition, and the receiving means of the applicator may be configured to be removably attachable to the intermediate member.

Preferably, such applicators are provided in individual, sealed packaging.

According to a third aspect of the present invention, a kit is provided comprising a pharmaceutical composition according to the first aspect of the present application. The kit may further include an applicator, such as an applicator according to the second aspect of the invention.

In one embodiment, the kit comprises a pharmaceutical composition for rapid administration of a therapeutic agent, and a pharmaceutical composition for sustained administration of the same or a different therapeutic agent, at least one of the compositions being in accordance with the first aspect of the present invention. The kit may comprise two or more compositions in accordance with the first aspect of the present invention, providing both rapid and sustained transdermal administration.

Alternatively, such a kit could comprise a combination of a transdermal composition according to the present invention and a therapeutic agent formulated for administration via another route, for example an oral dosage form.

Such kits would be of importance in conditions where sustained administration of the therapeutic agent is required, but where it is also desirable to rapidly and transiently raise blood levels of the therapeutic agent, for example in order to alleviate worsening of symptoms or specific “episodes”.

In one embodiment, the kit comprises at least one dose of the pharmaceutical composition for sustained transdermal administration, and more than one dose of the pharmaceutical composition for rapid transdermal administration, for administration at regular intervals throughout the period for which one sustained transdermal composition is intended. The number of doses of the pharmaceutical composition for rapid administration and for sustained administration may be provided in accordance with medical recommendations.

In one embodiment, the kit comprises at least one dose of the pharmaceutical composition for sustained transdermal administration, and a sufficient number of doses of the pharmaceutical composition for rapid transdermal administration to be taken at regular intervals throughout the period for which one sustained transdermal composition is intended, and one or more applicators according to the second aspect of the present invention.

Kits in accordance with the present invention can include instructions for use.

The present invention will now be illustrated by the following Examples, which do not limit the invention in any way.

### EXAMPLE 1

| Ingredients: | % w/w |
|---|---|
| Softisan 133 | 80 |
| Migylol 812 N | 5 |
| Lexol IPM | 2.5 |
| Fitoderm | 1.5 |
| Dry Flo AF Pure | 10 |
| Diclofenac sodium | 1 |

### EXAMPLE 2

| Ingredients: | % w/w |
|---|---|
| Softisan 142 | 80 |
| Migylol 812 N | 5 |
| Lexol IPM | 2.5 |
| Fitoderm | 1.5 |
| Dry Flo AF Pure | 10 |
| Diclofenac | 1 |

### EXAMPLE 3

| Ingredients: | % w/w |
|---|---|
| Softisan 142 | 79 |
| Migylol 812 N | 5 |
| Lexol IPM | 2.5 |
| Fitoderm | 1.5 |
| Dry Flo AF Pure | 10 |
| Propylene Glycol | 1 |
| Diclofenac | 1 |

### Method of Preparation for Examples 1-3

All ingredients excluding the diclofenac and dry flo were melted down until molten, and the temperature of the bulk was maintained at 60° C. The diclofenac and dry flo were carefully sheared into the bulk using a Silverson mixer. The bulk was solidified by exposing to low temperature, for example, 4° C. The solidified bulk was milled down and granulated also at low temperature, for example, 4° C.

### EXAMPLE 4

| Ingredients: | % w/w |
|---|---|
| Softisan 142 | 78 |
| Migylol 812 N | 5 |
| Lexol IPM | 2.5 |
| Fitoderm | 1.5 |
| Dry Flo AF Pure | 10 |
| Ethanol | 1 |
| Propylene glycol | 1 |
| Diclofenac sodium | 1 |

### EXAMPLE 5

| Ingredients: | % w/w |
|---|---|
| Softisan 133 | 79 |
| Migylol 812 N | 5 |
| Lexol IPM | 2.5 |
| Fitoderm | 1.5 |
| Dry Flo AF Pure | 10 |
| Ethanol | 1 |
| Diclofenac sodium | 1 |

### EXAMPLE 6

| Ingredients: | % w/w |
|---|---|
| Softisan 142 | 79 |
| Migylol 812 N | 5 |
| Lexol IPM | 2.5 |
| Fitoderm | 1.5 |
| Dry Flo AF Pure | 10 |
| Ethanol | 1 |
| Diclofenac sodium | 1 |

### Method of Preparation for Examples 4-6

All ingredients excluding the diclofenac, dry flo and ethanol were melted down until molten, and the temperature of the bulk was maintained at 60° C. The diclofenac and dry flo were carefully sheared into the bulk using a Silverson mixer. Once the temperature of the bulk had reduced to a suitably low temperature temperature (for example, less than 20° C.), the ethanol was mixed in. The bulk was solidified by exposing to low temperature, for example 4° C. The solidified bulk was milled down and granulated also at low temperature, for example 4° C.

### EXAMPLE 7

| Ingredients: | % w/w |
|---|---|
| Fentanyl | 0.72 |
| Softisan 133 | 83.28 |
| Dry Flo AF Pure | 10 |
| Migylol 812N | 5 |
| Fitoderm (veg squalene) | 1 |

### Method of Preparation for Example 7

All ingredients excluding the fentanyl and dry flo were melted down until molten, and the temperature of the bulk was then maintained at 60° C. The fentanyl and dry flo were carefully sheared into the bulk using a Silverson mixer. The bulk was solidified by exposing to low temperature, for example 4° C. The solidified bulk was milled down and granulated also at low temperature, for example, 4° C.

Percentages are by weight based on the total weight of the combined ingredients.

1. A pharmaceutical composition for topical application comprising a therapeutic agent and a pharmaceutically acceptable carrier, for transdermal administration of the therapeutic agent to provide a local therapeutic effect.

2. A pharmaceutical composition as claimed in claim 1, wherein the composition also provides a systemic therapeutic effect.

3. A pharmaceutical composition as claimed in claim 1, wherein the therapeutic agent is a selected from the group of agents known for the treatment of pain or inflammation or is a local anaesthetic.

4. A pharmaceutical composition as claimed in claim 3, wherein the therapeutic agent is diclofenac.

5. A pharmaceutical composition as claimed in claim 1, wherein the carrier comprises a glyceride, cocoa butter, theobroma, a high molecular weight polyethylene glycol, a polyoxyethylene, lanolin or a derivative thereof, a fatty acid, fatty alcohol or fatty ester, or an organic oil.

6. A pharmaceutical composition as claimed in claim 1, wherein the composition is substantially free of preservatives.

7. A pharmaceutical composition as claimed in claim 1, wherein the composition further comprises one or more preservatives to prevent or reduce contamination of the composition during preparation.

8. A pharmaceutical composition as claimed in claim 1, wherein the composition is substantially free of antioxidants.

9. A pharmaceutical composition as claimed in claim 1, wherein the carrier constitutes not less than about 60% by weight based on the weight of the pharmaceutical composition.

10. A pharmaceutical composition as claimed in claim 1, wherein the composition further comprises one or more solvents.

11. A pharmaceutical composition as claimed in claim 10, wherein the solvent comprises ethanol, menthol, thymol, eucalyptol, eucalyptus oil, benzyl alcohol, isopropyl alcohol, propylene glycol, methylated spirit, phenol, cyclodextrins, ethyl oleate, eugenol, glycerol, levomenol, monoethanolamine oleate, myristyl alcohol, octyldodecanol, methyl alcohol, coconut oil or silicone oil.

12. A pharmaceutical composition as claimed in claim 1, wherein the composition is spreadable.

13. A pharmaceutical composition as claimed in claim 12, wherein the composition is in the form of an ointment, cream or gel.

14. A pharmaceutical composition as claimed in claim 1, wherein the composition is substantially solid at a temperature of about 25° C. or less and has a softening point of not higher than the skin temperature of a mammalian patient.

15. A pharmaceutical composition as claimed in claim 1, further comprising a means of substantially occluding the pharmaceutical composition from the air upon application.

16. A pharmaceutical composition as claimed in claim 1, wherein the composition provides transdermal administration of the therapeutic agent within a period of 20 minutes or less from topical application to a subject.

17. A pharmaceutical composition as claimed in claim 16, wherein the composition provides transdermal administration of the therapeutic agent within a period of no more than 15, 10, 5, 4, 3, 2 minutes, 1 minute or 30 seconds from topical application to a subject.

18. A pharmaceutical composition as claimed in claim 1, providing transdermal administration of the therapeutic agent over a period of at least 30 minutes from topical application to the subject.

19. A pharmaceutical composition as claimed in claim 18, wherein the composition provides transdermal administration of the therapeutic agent over a period of at least about 1, 2, 4, 8, 12, 24, 48 or 72 hours from topical application.

20. A medicinal plaster comprising a pharmaceutical composition as claimed in claim 1.

21. An applicator for applying a pharmaceutical composition as claimed in claim 1.

22. A kit comprising a pharmaceutical composition according to claim 1.

23. Use of a therapeutic agent in the manufacture of a medicament comprising a composition as claimed in claim 1.

24. A use as claimed in claim 23, wherein the medicament is for treating pain or inflammation, or for providing local anaesthesia.

25. A pharmaceutical composition or product substantially as described in any one of the Examples.
